(19) United States
(12) Patent Application Publication
(10) Pub. No.: US 2022/0126075 A1
(43) Pub. Date: Apr. 28, 2022

(54) Microrobot Controlling Drug Release By Sound Waves And Method Of Manufacturing The Microrobot

(71) Applicant: Daegu Gyeongbuk Institute of Science and Technology, Daegu (KR)

(72) Inventors: Hong Soo Choi, Daegu (KR); Seung Min Noh, Gwangju (KR); Jin Young Kim, Daegu (KR); Jong Eon Park, Daejeon (KR)

(73) Assignee: Daegu Gyeongbuk Institute of Science and Technology, Daegu (KR)

(21) Appl. No.: 17/500,331

(22) Filed: Oct. 13, 2021

(30) Foreign Application Priority Data
Oct. 27, 2020 (KR) 10-2020-0139919

(51) Int. Cl. A61M 31/00; A61M 37/00; B29C 35/08

(52) U.S. Cl. CPC A61M 31/002; A61M 37/0092; B29K 2105/0035; A61M 2205/0244; A61M 2207/00; B29C 35/0805

(57) ABSTRACT

A microrobot of which a drug release is controlled by a sound wave applied from outside, and a method of manufacturing the microrobot are disclosed. The method includes mixing and storing magnetic nanoparticles and a drug in a biodegradable resist, and forming a microrobot having a three-dimensional (3D) porous structure at the resist through two-photon polymerization (TPP). The microrobot is formed to control a release rate of the drug stored in the resist by a sound wave applied from the outside.

### CROSS-REFERENCE TO RELATED APPLICATION

This application claims the benefit of Korean Patent Application No. 10-2020-0139919 filed on Oct. 27, 2020, and in the Korean Intellectual Property Office, the entire disclosure of which is incorporated herein by reference for all purposes.

### BACKGROUND

### 1. Field of the Invention

One or more example embodiments relate to a microrobot controlling drug release by a sound wave applied from outside.

### 2. Description of the Related Art

A drug delivery system (DDS) is a system designed to efficiently deliver a required amount of a drug to minimize side effects and maximize the efficacy and effectiveness of the drug. Methods of delivering drugs may be classified by delivery route, drug type, and delivery technology form. The methods include, for example, an injection and infusion method developed in the 1960s, a suppository method developed in the 1970s, a nasal and oral administration method developed in the 1980s, and a direct delivery method of delivering a drug directly to the skin, lungs, and mouth developed in the 1990s. The DDS may reduce the time and cost needed to develop a new drug and be highly likely to be successful, and has thus become one of the advanced technologies into which developed countries started conducting active research from the 1970s. Korea started the research in earnest back in the 1990s.

The DDS may include a sustained release DDS (SRDDS), a controlled release DDS (CRDDS), and a targeted DDS (TDDS). The SRDDS may use a formulation designed to reduce a drug release rate to prevent low bioavailability or prevent extremely slow drug absorption or extremely fast drug excretion. The CRDDS may be designed to control an actual therapeutic effect by controlling a concentration of a target site which is mainly plasma, and may extend a drug delivery time and reproduce and predict a drug release rate as the SRDDS does. The TDDS may inhibit a non-specific distribution such that a drug is selectively delivered only to a cancer cell when a chemotherapeutic agent is used because the agent may be highly toxic even to a normal cell, thereby protecting a non-target site and delivering a drug only to a target site.

The TDDS may deliver a drug to a target site using nanorobots or microrobots. Currently, fundamental research is being actively conducted on the use of micro/nanotechnology-based micro/nanorobotics for medical purposes. For example, in the case of anticancer drugs, a large-scale market is expected to be formed, and cancer treatment methods using existing anticancer drugs have some issues such as non-selective toxicity and related side effects due to low targeted performance. Thus, there is active research on such a micro/nanotechnology-based DDS that may minimize side effects and maximize the efficacy of existing drugs and on the commercialization of the DDS. In addition, over the last few decades, biocompatible polymer-based nanoparticles have been developed as a representative targeted drug delivery platform, and various studies have been conducted to improve drug solubility, improve drug content while preventing drug loss, increase target delivery ability, and enhance drug release properties at an active site.

Recently, active research is being conducted to improve in-vitro or in-vivo drug delivery performance and effects of anticancer drugs by developing various types of intelligent nanoparticle formulations that release a drug in response to various environments (e.g., pH, oxidation-reduction reaction, temperature, magnetic field, light, etc.) of a living body.

The above description is information the inventor(s) acquired during the course of conceiving the present disclosure, or already possessed at the time, and is not necessarily art publicly known before the present application was filed.

### SUMMARY

Example embodiments provide a microrobot and a method of manufacturing the microrobot that enable a systematic and effective drug treatment by controlling a release of a drug using a sound wave applied from outside.

Additional aspects of example embodiments are not limited to what is described in the foregoing, and other aspects that are not described above may also be learned by those skilled in the art from the following description.

A microrobot and a method of manufacturing the microrobot will be described according to an example embodiment.

According to an aspect, there is provided a method of manufacturing a microrobot, mixing and storing magnetic nanoparticles and a drug in a biodegradable resist, and forming a microrobot having a three-dimensional (3D) porous structure at the resist through two-photon polymerization (TPP). The microrobot may control a release rate of the drug stored in the resist by a sound wave applied from outside.

As acoustic streaming and stable cavitation occur in the microrobot by the sound wave applied from the outside, the resist may be decomposed. The microrobot may have a 3D helix structure.

According to another aspect, there is provided a microrobot, including a body formed to have a 3D porous structure by storing a drug and magnetic nanoparticles in a biodegradable resist. The body may control a release rate of the drug stored in the resist by a sound wave applied from outside.

The body may have a 3D helix structure.

According to example embodiments, irradiating a sound wave and releasing a drug when a microrobot reaches a target position may prevent adverse effects of the drug from occurring as the drug is released while the microrobot is moving. In addition, controlling the release of the drug at the target position and based on a required treatment condition may enable a more efficient and systematic drug treatment.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIG. 1 is a perspective view illustrating a microrobot according to an example embodiment;

FIG. 2 is a diagram illustrating an operation of releasing a drug from the microrobot in FIG. 1;

FIG. 3 is a diagram illustrating a detailed structure of the microrobot in FIG. 2;

FIG. 4 is a diagram illustrating a method of manufacturing a microrobot according to an example embodiment; and

FIGS. 5A and 5B are graphs illustrating a polymer degradation amount and a drug release amount when a sound wave is applied by a microrobot according to example embodiments.

### DETAILED DESCRIPTION

Hereinafter, example embodiments will be described in detail with reference to the accompanying drawings. It should be understood, however, that there is no intent to limit this disclosure to the particular example embodiments disclosed. On the contrary, example embodiments are to cover all modifications, equivalents, and alternatives falling within the scope of the example embodiments.

The terminology used herein is for the purpose of describing particular example embodiments only and is not to be limiting of the example embodiments. As used herein, the singular forms “a,” “an,” and “the,” are intended to include the plural forms as well, unless the context clearly indicates otherwise. As used herein, the term “and/or” includes any one and any combination of any two or more of the associated listed items. It will be further understood that the terms “comprises” and/or “comprising,” when used in this specification, specify the presence of stated features, integers, steps, operations, elements, components or a combination thereof, but do not preclude the presence or addition of one or more other features, integers, steps, operations, elements, components, and/or groups thereof.

Unless otherwise defined herein, all terms used herein including technical or scientific terms have the same meanings as those generally understood by one of ordinary skill in the art. Terms defined in dictionaries generally used should be construed to have meanings matching contextual meanings in the related art and are not to be construed as an ideal or excessively formal meaning unless otherwise defined herein.

When describing the examples with reference to the accompanying drawings, like reference numerals refer to like constituent elements and a repeated description related thereto will be omitted. In the description of examples, detailed description of well-known related structures or functions will be omitted when it is deemed that such description will cause ambiguous interpretation of the present disclosure.

In addition, terms such as first, second, A, B, (a), (b), and the like may be used herein to describe components. Each of these terminologies is not used to define an essence, order, or sequence of a corresponding component but used merely to distinguish the corresponding component from other component(s). When it is mentioned that one component is “connected” or “accessed” to another component, it may be understood that the one component is directly connected or accessed to another component or that still other component is interposed between the two components. It should be noted that if it is described in the specification that one component is “directly connected” or “directly joined” to another component, still other component may not be present therebetween.

Example embodiments will be described in detail with reference to the accompanying drawings. When describing the example embodiments with reference to the accompanying drawings, like reference numerals refer to like components and a repeated description related thereto will be omitted.

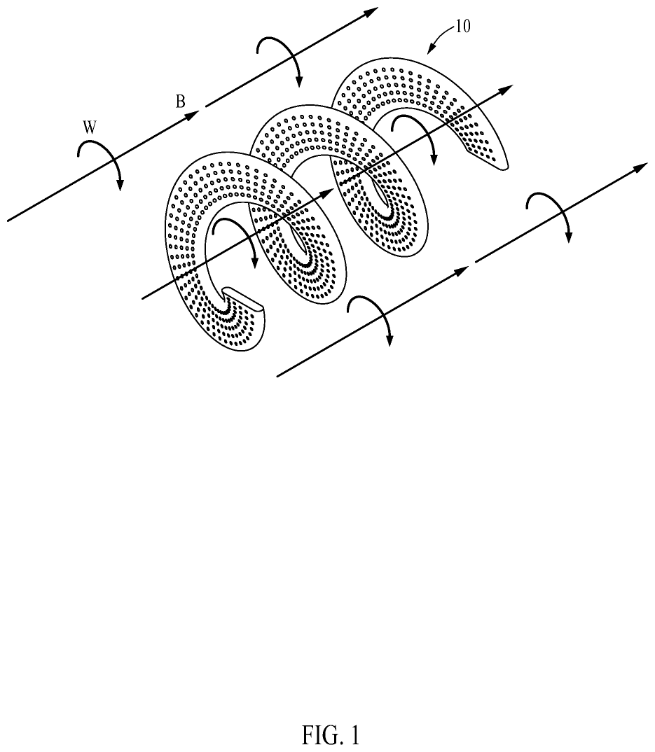

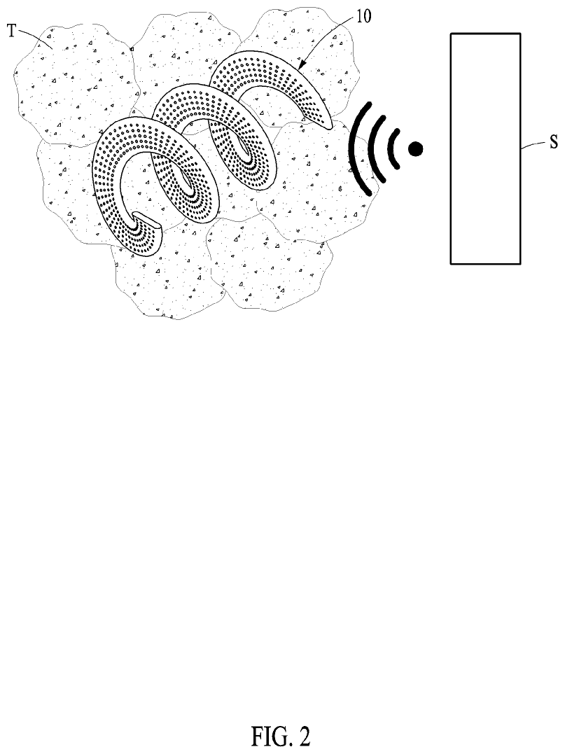

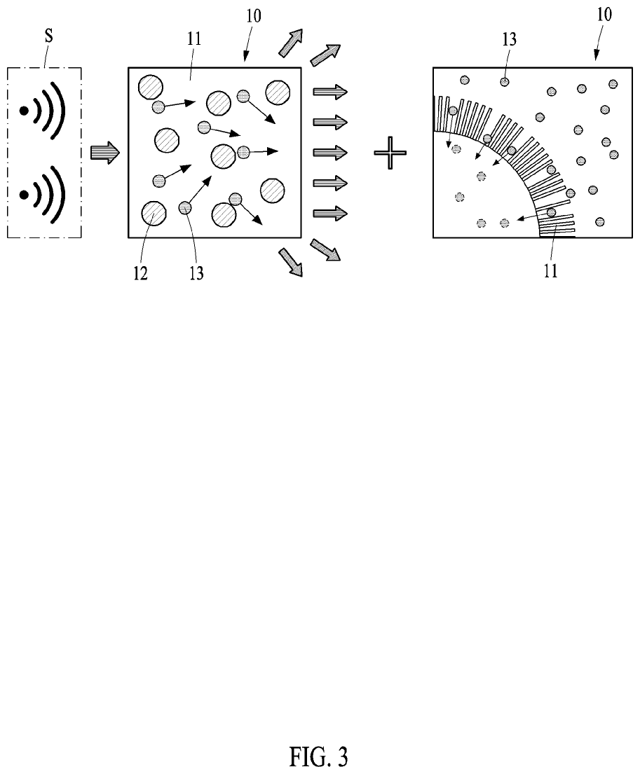

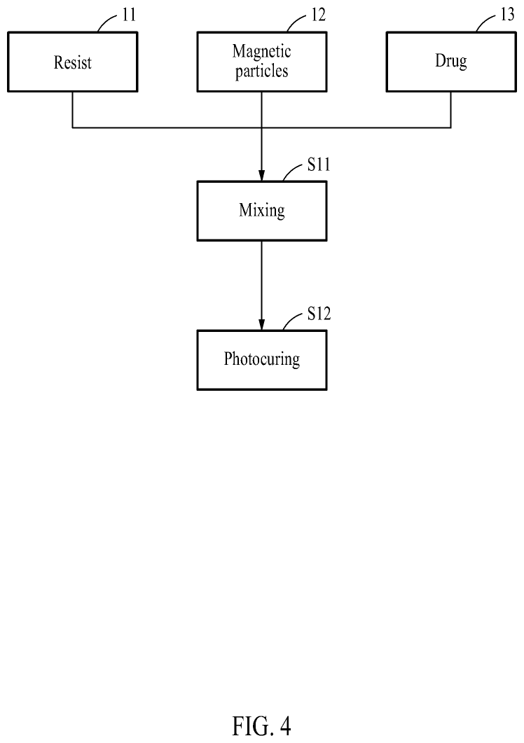

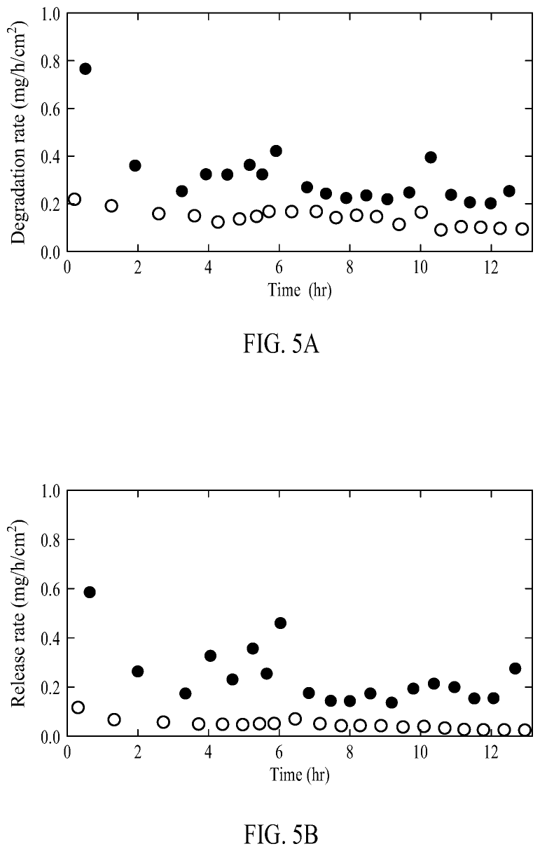

Hereinafter, a microrobot 10 and a method of manufacturing the microrobot 10 will be described with reference to FIGS. 1 through 5. FIG. 1 is a perspective view illustrating the microrobot 10, FIG. 2 is a diagram illustrating an operation of releasing a drug 13 at a target T from the microrobot 10 of FIG. 1, FIG. 3 is a diagram illustrating a detailed structure of the microrobot 10 of FIG. 2, and FIG. 4 is a diagram illustrating a method of manufacturing the microrobot 10. FIGS. 5A and 5B are graphs illustrating a polymer degradation amount and a drug release amount when a sound wave is applied by the microrobot 10.

A microrobot used herein may refer to a micro-sized robot used for medical purposes, but is not limited thereto. The microrobot may also be a robot of nano or smaller size.

Referring to the accompanying drawings, the microrobot 10 may be formed by mixing a drug 13 and magnetic nanoparticles 12 in a biodegradable resist 11.

A body of the microrobot 10 may have a three-dimensional (3D) shape and a porous structure. For example, the microrobot 10 may be formed to have a 3D porous structure using a two-photon polymerization (TPP) method. Using the TPP method, the microrobot 10 may have a high resolution and an ultrafine size.

In addition, the microrobot 10 may have a 3D helix structure. However, the shape of the microrobot 10 is not limited to the foregoing examples, and the microrobot 10 may be provided in various shapes of a 3D porous structure, such as, for example, a cylinder, a hexahedron, a sphere, and an oval.

The magnetic nanoparticles 12 may provide a driving force that allows the microrobot 10 to move and rotate the microrobot 10 to the target T by a magnetic field applied from outside. The magnetic nanoparticles 12 may be biocompatible particles and use, for example, iron oxide particles including Fe₃O4.

The drug 13 may be a drug or medicine to act on the target T, and be selected according to a type of the target T.

The drug 13 may be released as the resist 11 is decomposed when the microrobot 10 reaches the target T and a sound wave is applied from the outside.

Referring to FIG. 3, when a sound wave S is applied to the microrobot 10, acoustic streaming and stable cavitation may occur in the microrobot 10 and the resist 11, and a physical stress may be applied to the microrobot 10. Such stress may allow the resist 11 to be decomposed, and the drug 13 stored or deposited therein may be released. In addition, sonoporation may occur in cells near a position of the target T by the sound wave S and microcracks may be generated. Thus, the drug 13 released from the microrobot 10 may be more effectively absorbed into cells of the target T, improving a treatment effect of the drug 13.

The microrobot 10 has a large surface area due to its 3D porous structure, and thus an amount of the drug 13 released by the sound wave S may be great. In addition, a release rate and a released amount of the drug 13 may be adjusted by controlling an intensity of the sound wave S to be applied to the microrobot 10.

FIGS. 5A and 5B are graphs illustrating a polymer degradation amount and a drug release amount when a sound wave is applied and when the sound wave is not applied. In FIGS. 5A and 5B, black dots represent a case in which a sound wave is applied, and white dots represent a case in which the sound wave is not applied. Referring to FIGS. 5A and 5B, when the sound wave is applied, polymer erosion may occur due to cavitation generated in the microrobot 10, and thus the drug 13 may be rapidly released.

The microrobot 10 may be formed by mixing the drug 13 and the magnetic nanoparticles 12 in the biodegradable resist 11 that is photocurable, and photocuring (or performing polymerization on) the mixture.

Hereinafter, a method of manufacturing the microrobot 10 will be described with reference to FIG. 4.

In step S11, the drug 13 may be stored or deposited in the photocurable and biodegradable resist 11 and mixed with the magnetic nanoparticles 12.

In step S12, the resist 11 may be irradiated with light to be formed into a predetermined shape and cured to form the microrobot 10.

The microrobot 10 may have a porous structure and a 3D shape.

The microrobot 10 may be formed to have a 3D porous structure using a TPP method. The microrobot 10 may be formed to have high resolution and an ultrafine size using the TPP method.

However, the method of manufacturing the microrobot 10 may use other various methods to form a 3D porous structure of a micro or nano size, for example, 3D printing or lithography, in addition to the TPP method.

According to example embodiments, the drug 13 may be stored or deposited in the resist 11 of the microrobot 10 and may not be released while the microrobot 10 is moving in the body. Thus, it is possible to prevent adverse effects from occurring. In addition, the microrobot 10 may release the drug 13 when a sound wave S is applied, and thus release the drug 13 at an accurate position of a target T. The microrobot 10 may also control a release amount of the drug 13 based on an intensity of the sound wave S applied, and thereby adjust a release amount and a release rate of the drug 13 based on a required treatment condition. Thus, the microrobot 10 may enable a more efficient and systematic drug treatment.

The microrobot 10 may include the magnetic nanoparticles 12 and thus allow a 3D position control through a magnetic field applied from the outside. In addition, the microrobot 10 may wirelessly move into the position of the target T in the body, enabling a safe and precise movement. Further, the microrobot 10 may be formed with a biodegradable material and not include conventionally used metallic materials, for example, nickel and titanium, and thus be decomposed inside the body without a need to be retrieved after the use.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. A method of manufacturing a microrobot, comprising:
mixing and storing magnetic nanoparticles and a drug in a biodegradable resist; and
forming a microrobot having a three-dimensional (3D) porous structure at the resist through two-photon polymerization (TPP),
wherein the microrobot is configured to control a release rate of the drug stored in the resist by a sound wave applied from outside.

2. The method of claim 1, wherein, as acoustic streaming and stable cavitation occur in the microrobot by the sound wave applied from the outside, the resist is decomposed.

3. The method of claim 1, wherein the microrobot has a 3D helix structure.

4. A microrobot, comprising:
a body formed to have a three-dimensional (3D) porous structure by storing a drug and magnetic nanoparticles in a biodegradable resist,
wherein the body is configured to control a release rate of the drug stored in the resist by a sound wave applied from outside.

5. The microrobot of claim 4, wherein the body has a 3D helix structure.
